SEQUENCE LISTING (32 sequences)

SEQ ID NO: 1 - protein, 15 aa, synthetic construct
GDPIADMIDQTVNNQ

SEQ ID NO: 2 - protein, 15 aa, synthetic construct
DMIDQTVNNQVNRSL

SEQ ID NO: 3 - protein, 15 aa, synthetic construct
TALQVLPTAADTEAS

SEQ ID NO: 4 - protein, 15 aa, synthetic construct
LPTAADTEASSHRLG

SEQ ID NO: 5 - protein, 15 aa, synthetic construct
TRCVLNHHSTQETAI

SEQ ID NO: 6 - protein, 15 aa, synthetic construct
YAQLRRKCELFTYMR

SEQ ID NO: 7 - protein, 15 aa, synthetic construct
DGYPTFGEHLQANDL

SEQ ID NO: 8 - protein, 15 aa, synthetic construct
FGEHLQANDLDYGQC

SEQ ID NO: 9 - protein, 15 aa, synthetic construct
SITLRVYMRIKHVRA

SEQ ID NO: 10 - protein, 15 aa, synthetic construct
DATAVDKPTRPDVSV

SEQ ID NO: 11 - protein, 15 aa, synthetic construct
ALLVAVLPEYVLGTI

SEQ ID NO: 12 - protein, 15 aa, synthetic construct
AGGTGNENSHPPYAT

SEQ ID NO: 13 - protein, 15 aa, synthetic construct
PYMNTVPFDSALNHC

SEQ ID NO: 14 - protein, 15 aa, synthetic construct
GIPTELKPGTNQFLT

SEQ ID NO: 15 - protein, 15 aa, synthetic construct
NQFLTTDDGVSAPIL

SEQ ID NO: 16 - protein, 15 aa, synthetic construct
TDDGVSAPILPGFHP

SEQ ID NO: 17 - protein, 15 aa, synthetic construct
SAPILPGFHPTPPIH

SEQ ID NO: 18 - protein, 15 aa, synthetic construct
PGFHPTPPIHIPGEV

SEQ ID NO: 19 - protein, 15 aa, synthetic construct
TPPIHIPGEVHNLLE

SEQ ID NO: 20 - protein, 15 aa, synthetic construct
IPGEVHNLLEICRVE

SEQ ID NO: 21 - protein, 15 aa, synthetic construct
HNLLEICRVETILEV

SEQ ID NO: 22 - protein, 15 aa, synthetic construct
MQRLCFPVSVQSKTG

SEQ ID NO: 23 - protein, 15 aa, synthetic construct
FRADPGRDGPWQSTI

SEQ ID NO: 24 - protein, 12 aa, synthetic construct
DTEDIEQTANIQ

SEQ ID NO: 25 - protein, 15 aa, synthetic construct
MSQDPKKFTDPVMDV

SEQ ID NO: 26 - protein, 15 aa, synthetic construct
KKFTDPVMDVIHEMA

SEQ ID NO: 27 - protein, 14 aa, synthetic construct
PVMDVIHEMAPPLK

SEQ ID NO: 28 - protein, 323 aa, Coxsackievirus A16
MGSQVSTQRSGSHENSNSASEGSTINYTTINYYKDAYAASAGRQDMSQDPKKFTDPVMDVIHEMAPPLKSPSAEACGYSDRVAQLTIGNSTITTQEAANIVIAYGEWPEYCPDTDATAVDKPTRPDVSVNRFFTLDTKSWAKDSKGWYWKFPDVLTEVGVFGQNAQFHYLYRSGFCVHVQCNASKFHQGALLVAVLPEYVLGTIAGGTGNENSHPPYATTQPGQVGAVLTHPYVLDAGIPLSQLTVCPHQWINLRTNNCATIIVPYMNTVPFDSALNHCNFGLLVVPVVPLDFNAGATSEIPITVTIAPMCAEFAGLRQAVKQ

SEQ ID NO: 29 - protein, 297 aa, Coxsackievirus A16
GDPIADMIDQTVNNQVNRSLTALQVLPTAADTEASSHRLGTGVVPALQAAETGASSNASDKNLIETRCVLNHHSTQETAIGNFFSRAGLVSIITMPTTGTQNTDGYVNWDIDLMGYAQLRRKCELFTYMRFDAEFTFVVAKPNGELVPQLLQYMYVPPGAPKPTSRDSFAWQTATNPSVFVKMTDPPAQVSVPFMSPASAYQWFYDGYPTFGEHLQANDLDYGQCPNNMMGTFSIRTVGTEKSPHSITLRVYMRIKHVRAWIPRPLRNQPYLFKTNPNYKGNDIKCTSTSRDKITTL

SEQ ID NO: 30 - protein, 254 aa, Coxsackievirus A16
SPSAEACGYSDRVAQLTIGNSTITTQEAANIVIAYGEWPEYCPDTDATAVDKPTRPDVSVNRFFTLDTKSWAKDSKGWYWKFPDVLTEVGVFGQNAQFHYLYRSGFCVHVQCNASKFHQGALLVAVLPEYVLGTIAGGTGNENSHPPYATTQPGQVGAVLTHPYVLDAGIPLSQLTVCPHQWINLRTNNCATIIVPYMNTVPFDSALNHCNFGLLVVPVVPLDFNAGATSEIPITVTIAPMCAEFAGLRQAVKQ

SEQ ID NO: 31 - protein, 242 aa, Coxsackievirus A16
GIPTELKPGTNQFLTTDDGVSAPILPGFHPTPPIHIPGEVHNLLEICRVETILEVNNLKTNETTPMQRLCFPVSVQSKTGELCAAFRADPGRDGPWQSTILGQLCRYYTQWSGSLEVTFMFAGSFMATGKMLIAYTPPGGNVPADRITAMLGTHVIWDFGLQSSVTLVVPWISNTHYRAHARAGYFDYYTTGIITIWYQTNYVVPIGAPTTAYIVALAAAQDNFTMKLCKDTEDIEQTANIQ

SEQ ID NO: 32 - protein, 69 aa, Coxsackievirus A16
MGSQVSTQRSGSHENSNSASEGSTINYTTINYYKDAYAASAGRQDMSQDPKKFTDPVMDVIHEMAPPLK

(19) United States
(12) Patent Application Publication
(10) Pub. No.: US 2025/0250327 A1
(43) Pub. Date: Aug. 7, 2025

(54) YOLK IMMUNOGLOBULIN (IgY) AGAINST COXSACKIEVIRUS A16 AND/OR ENTEROVIRUS A71 AND ITS PREPARATION AND USES THEREOF

(71) Applicants: NATIONAL HEALTH RESEARCH INSTITUTES, Miaoli County (TW); NATIONAL DEFENSE MEDICAL CENTER, Taipei City (TW)

(72) Inventors: Chia-Chyi LIU, Miaoli County (TW); Yu-Sheng SHEN, Miaoli County (TW); Chi-Hsun CHEN, Miaoli County (TW); Chia-Tsui YEH, Taipei City (TW); Cheng-Cheung CHEN, Taipei City (TW); Xin-An CHEN, Taipei City (TW)

(73) Assignees: NATIONAL HEALTH RESEARCH INSTITUTES, Miaoli County (TW); NATIONAL DEFENSE MEDICAL CENTER, Taipei City (TW)

(21) Appl. No.: 19/042,290

(22) Filed: Jan. 31, 2025

Related U.S. Application Data
(60) Provisional application No. 63/627,946, filed on Feb. 1, 2024.

(51) Int. Cl. C07K 16/10; A61K 39/00

(52) U.S. Cl. CPC C07K 16/1009; A61K 2039/505; C07K 2317/11; C07K 2317/33; C07K 2317/76

(57) ABSTRACT

The present invention relates to immunoglobulin yolk (IgY) against coxsackievirus A16 and/or enterovirus A71 and its preparation and uses thereof.

### RELATED APPLICATIONS

This application claims the benefit of U.S. provisional application No. 63/627,946, filed Feb. 1, 2024 under 35 U.S.C. § 119, the entire content of which is incorporated herein by reference.

### REFERENCE TO ELECTRONIC SEQUENCE LISTING

The application contains a Sequence Listing which has been submitted electronically in .XML format and is hereby incorporated by reference in its entirety. Said .XML copy, created on Jan. 23, 2025, is named “NHR0015US” and is 29,881 bytes in size. The sequence listing contained in this .XML file is part of the specification and is hereby incorporated by reference herein in its entirety.

### TECHNOLOGY FIELD

The present invention relates to immunoglobulin yolk (IgY) against coxsackievirus A16 and/or enterovirus A71 and its preparation and uses thereof.

### BACKGROUND OF THE INVENTION

Coxsackievirus A16 (CVA16) infection most commonly manifests as a childhood exanthema known as hand, foot, and mouth disease (HFMD) but has a tendency to cause neurological complications during acute infection. CVA16 infection is usually mild and not as severe as enterovirus A71 (EV-A71) in the clinical manifestations of HFMD, but CVA16 and other coxsackievirus A group viruses cause more HFMD epidemics than EV-A71 [Mao et al., 2014; Klein & Chong, 2015; Fang & Liu, 2018; Fang & Liu, 2022]. Since formalin-inactivated EV-A71 (FI-EV-A71) vaccines fail to induce cross-neutralization response against CVA16 [Chou et al., 2013; Fang & Liu, 2018]; therefore, antiviral drugs and available vaccines are needed for prevention of CVA16 infection. Several anti-CVA16 compounds were developed, but none the clinical trials of these compounds have been done for HFMD [Yuan et al., 2023]. Several CVA16 vaccine candidates have also been developed, including the formalin-inactivated CVA16 (FI-CVA16) vaccines and virus-like particle (VLP) vaccines [Fang & Liu, 2018; Fang & Liu, 2022]. The current development of CVA16 vaccine has been incorporated into the multivalent HFMD vaccines [Fang & Liu, 2018], and some clinical trials have begun in Korea and China.

CVA16 is a non-enveloped RNA virus, which belongs to the family Picornaviridae and genus Enterovirus A. The genome size of CVA16 is about 7.4 K base-pair that translates into one polyprotein (structural proteins: VP1 to VP4; non-structural proteins: 2A to 2C and 3A to 3D) [Solomon et al., 2010]. CVA16 was first isolated from South Africa in 1951 [Aswathyraj et al., 2016]. Several outbreaks of CVA16-related HFMD have also been reported in many countries including Australia, China, India, Japan, Korea, Singapore, Taiwan, Thailand, Malaysia, the United Kingdom and Vietnam [Mao et al., 2014; Aswathyraj et al., 2016; Tikute & Lavania, 2023]. Based on the genomic sequence of VP1 protein, CVA16 strains are classified into three groups (A, B, and C), and further subclassified into different subgenogroups (B1a to B1c, B2a to B2c and C1 to C3) [Yu et al., 2016]. To evaluate the immunogenicity of CVA16 vaccine candidates, mouse, rat, and monkey were mostly used in the reported studies [Fang & Liu, 2018].

Immunoglobulin Y (IgY) is the major antibody in chickens, which is considered as functionally equivalent to the immunoglobulin G (IgG) of mammalian. A large amount of IgY can be obtained from chicken egg yolk of immunized animals, which is non-invasive for animal and cost-effective for antibody production. Many IgY-based products have been commercialized [Leslie and Clem 1969; Yakhkeshi et al., 2022; Wang et al., 2023; Suresh et al., 2023]. IgY-based products has developed rapidly with broad diagnostic, preventive, and therapeutic applications in research and clinic, but few human clinical trials have been reported. Recently, passive immunization with IgY has been tested for the treatment of viral respiratory infections, such as influenza, SARS-COV-2, etc. [Yang et al., 2014; Yeh et al., 2022; Wang et al., 2023;]. In past enterovirus studies, the passive protection of IgY or monoclonal single-chain variable fragments (scFv) were shown to prevent enterovirus A71 (EV-A71) and CVA16-related HFMD [Liou et al., 2010; Nie et al., 2019; Gao et al., 2019; Mwale et al., 2021; Lee et al., 2022]. Anti-EV-A71 IgY and Anti-CVA16 IgY were induced with viral extracts.

There is still a need to prepare and provide an antibody against CVA16 and/or EV-A71 for the purposes of treatment, prevention and/or diagnosis.

### SUMMARY OF THE INVENTION

The present invention relates to immunoglobulin yolk (IgY) against coxsackievirus A16 (CVA16) and/or enterovirus A71 (EV-A71) and its preparation and uses thereof.

In one aspect, the present invention relates to a method for preparing an IgY against CVA16 comprising
(i) immunizing an egg-laying avian with CVA16 particles selected from the group consisting of CVA16 empty (E) particles, CVA16 full (F) particles and a combination thereof to obtain an immunized egg product from the avian; and optionally
(ii) extracting and purifying an anti-CVA16 IgY from the immunized egg product.

In some embodiments, the CVA16 particles are purified.

In some embodiments, the CVA16 particles are CVA16 empty (E) particles

In some embodiments, the CVA16 particles are CVA16 full (F) particles.

In some embodiments, the CVA16 particles are CVA16 E particles and CVA16 F particles.

In some embodiments, the avian is selected from the group consisting of chicken, turkey, goose, duck, pheasant, quail, pigeon and ostrich.

In some embodiments, the avian is chicken.

In some embodiments, the CVA16 particles are formulated with an adjuvant.

In some embodiments, the avian was administered with a first injection, followed by one or more booster injections, of the CVA16 particles.

In some embodiments, the CVA16 particles are CVA16 E particles and the anti-CVA16 IgY thus prepared neutralizes CVA16 and enterovirus A71 (EV-A71).

In some embodiments, the CVA16 particles are CVA16 F particles and the anti-CVA16 IgY thus prepared neutralizes CVA16 and fail to neutralize enterovirus A71 (EV-A71).

In another aspect, the present invention provides an anti-CVA16 IgY prepared by a method as described herein.

In some embodiments, the IgY of the present invention specifically binds to
(i) an epitope of capsid protein VP1 selected from the group consisting of VP1-1, VP1-2, VP1-5, VP1-6, VP1-14, VP-24, VP1-42, VP1-43, VP1-50 and any combination thereof, as shown in Table 2;
(ii) an epitope of capsid protein VP2 selected from the group consisting of VP2-10, VP2-25, VP2-28, VP2-40 and any combination thereof, as shown in Table 2;
(iii) an epitope of capsid protein VP3 selected from the group consisting of VP3-1, VP3-3, VP3-4, VP3-5, VP3-6, VP3-7, VP3-8, VP3-9, VP3-14, VP3-18, VP3-47 and any combination thereof, as shown in Table 2; and/or
(iv) an epitope of capsid protein VP4 selected from the group consisting of VP4-10, VP4-11, VP4-12 and any combination thereof, as shown in Table 2.

In some embodiments, the IgY of the present invention specifically binds to
(i) an epitope of capsid protein VP1 selected from the group consisting of VP1-1, VP1-2, VP1-5, VP1-42, VP1-50 and any combination thereof;
(ii) an epitope of capsid protein VP2 selected from the group consisting of VP2-25; and/or
(iii) an epitope of capsid protein VP3 selected from the group consisting of VP3-1, VP3-4, VP3-5, VP3-6, VP3-7, VP3-8, VP3-9, VP3-14 and any combination thereof.

In some embodiments, the IgY of the present invention does not bind to
(i) an epitope of capsid protein VP1 selected from the group consisting of VP1-6, VP1-14, VP-24, VP1-43 and any combination thereof;
(ii) an epitope of capsid protein VP2 selected from the group consisting of VP2-10, VP2-28, VP2-40 and any combination thereof;
(iii) an epitope of capsid protein VP3 selected from the group consisting of VP3-3, VP3-18, VP3-47 and any combination thereof; and/or
(iv) an epitope of capsid protein VP4 selected from the group consisting of VP4-10, VP4-11, VP4-12 and any combination thereof.

In some embodiments, the anti-CVA16 IgY neutralizes CVA16 and enterovirus A71 (EV-A71).

In some embodiments, the IgY of the present invention specifically binds to
(i) an epitope of capsid protein VP1 selected from the group consisting of VP1-1, VP1-6, VP1-14, VP-24, VP1-42, VP1-43 and any combination thereof;
(ii) an epitope of capsid protein VP2 selected from the group consisting of VP2-10, VP2-25, VP2-28, VP2-40 and any combination thereof;
(iii) an epitope of capsid protein VP3 selected from the group consisting of VP3-3, VP3-4, VP3-18, VP3-47 and any combination thereof; and/or
(iv) an epitope of capsid protein VP4 selected from the group consisting of VP4-10, VP4-11, VP4-12 and any combination thereof.

In some embodiments, the IgY of the present invention does not bind to
(i) an epitope of capsid protein VP1 selected from the group consisting of VP1-2, VP1-5, VP1-50 and any combination thereof; and/or
(ii) an epitope of capsid protein VP3 selected from the group consisting of VP3-1, VP3-5, VP3-6, VP3-7, VP3-8, VP3-9, VP3-14 and any combination thereof.

In some embodiments, the IgY of the present invention neutralizes CVA16 but fail to neutralize enterovirus A71 (EV-A71).

The present invention also provides a composition comprising an anti-CVA16 IgY as described herein and a carrier. The composition of the present invention can be a pharmaceutical composition or a food composition, for example.

In a further aspect, the present invention provides a method for treating or preventing CVA16 and/or EV-A71 infection, comprising administering to a subject in need thereof an effective amount of an anti-CVA16 IgY as described herein or a composition thereof.

In still a further aspect, the present invention provides a method for diagnosing CVA16 and/or EV-A71 infection, comprising the steps of
(i) providing a sample e.g. derived from a subject in need thereof;
(ii) reacting the sample with an anti-CVA16 IgY any of claims 1 to 19 or a composition of claim 20; and
(iii) detecting an antigen-antibody reaction between the anti-CVA16 IgY and CVA16 and/or EV-A71 present in the sample.

The details of one or more embodiments of the invention are set forth in the description below. Other features or advantages of the present invention will be apparent from the following detailed description of several embodiments, and also from the appending claims.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The foregoing summary, as well as the following detailed description of the invention, will be better understood when read in conjunction with the appended drawings. For the purpose of illustrating the invention, there are shown in the drawings embodiments which are presently preferred. It should be understood, however, that the invention is not limited to the precise arrangements and instrumentalities shown.

In the drawings:

FIG. 1 shows the protein profiles of purified CVA16 viral particles. The CVA16 empty (E) particle (fraction #06 and #07) and full (F) particle (fraction #13 and #14) were purified using sucrose gradient ultracentrifugation. Viral protein composition of E-particle: VP0+VP1+VP3; and viral protein composition of F-particle: VP1+VP2+VP3+VP4+viral RNA.

FIG. 2 shows the process for production of chicken egg yolk immunoglobulin. Chickens were immunized with inactivated viral particles. Four weeks after the immunization of laying hens by the 1st injection of the antigen proteins, a 2nd immunization was followed by the same procedures. Four weeks after the final injection, eggs were harvested and stored at 4° C. The purified IgYs were extracted from egg yolks.

FIG. 3 shows the protein profiles of the purified anti-CVA16 IgYs analyzed by SDS-PAGE (upper panel) and western blot (lower panel). The purified IgY of two chicken eggs in each group were confirmed after purification. The SDS-PAGE was performed by Coomassie blue stain. The western blot was performed by rabbit anti-chicken IgY HRP (primary antibody).

FIG. 4 shows the antigen reorganization of anti-CVA16 IgYs and anti-EV-A71 IgY analyzed by western blotting. Anti-CVA16-E IgY is a combination of A16-E1 and A16-E2 IgYs. Anti-CVA16-F IgY is a combination of A16-F1 and A16-F2 IgYs. M: protein marker; Lane 1: CVA16 E-particle 1 μg; Lane 2: CVA16 E-particle 0.5 μg; Lane 3: CVA16 E-particle 0.1 μg; Lane 4: CVA16 F-particle 1 μg; Lane 5: CVA16 F-particle 0.5 μg; Lane 6: CVA16 F-particle 0.1 μg, Lane 7: EV-A71 bulk 1 μg; Lane 8: EV-A71 bulk 0.5 μg; and Lane 9: EV-A71 bulk 0.1 μg.

FIG. 5 shows the antibody specificity assay of anti-CVA16 IgYs. Anti-CVA16-E IgY represents a combination of A16-E1 and A16-E2 IgYs. Anti-CVA16-F IgY represents a combination of A16-F1 and A16-F2 IgYs. Enterovirus antigens are coated in 96-well plate with 10 ng/well.

FIGS. 6A-6C show the liner epitope mapping of anti-CVA16-IgYs. A panel of 165 overlapping peptides covering the entire sequences of VP1 (57×15-mer peptides), VP2 (49×15-mer peptides), and VP3 (47×15-mer peptides), and VP4 (12×15-mer peptides) of CVA16 was used in peptide ELISA format for epitope mapping against (FIG. 6A) anti-CVA16-E IgY, (FIG. 6B) anti-CVA16-F IgY, and (FIG. 6C) anti-EV-A71 IgY.

FIG. 7 shows the proposed locations of CVA16 immunodominant epitopes based on sequence alignment and molecular modeling of CVA16 structure. The molecular structures of the two units of VP1, VP2 and VP3 are represented by different color, respectively. The peptide sequences that specific react with anti-CVA16 IgYs were labeled by yellow color. The red circle was predicted to be the three-dimensional configuration of the binding region with anti-CVA16 IgYs.

### DETAILED DESCRIPTION OF THE INVENTION

Unless defined otherwise, all technical and scientific terms used herein have the same meaning as commonly understood by a person skilled in the art to which this invention belongs.

As used herein, the singular forms “a”, “an”, and “the” include plural referents unless the context clearly dictates otherwise. Thus, for example, reference to “a component” includes a plurality of such components and equivalents thereof known to those skilled in the art.

The term “comprise” or “comprising” is generally used in the sense of include/including which means permitting the presence of one or more features, ingredients or components. The term “comprise” or “comprising” encompasses the term “consists” or “consisting of.”

As used herein, the term “antibody” (interchangeably used in plural form, antibodies) means an immunoglobulin molecule having the ability to specifically bind to a particular target antigenic molecule. As used herein, the term “antibody” includes not only intact (i.e., full-length) antibody molecules but also antigen-binding fragments thereof retaining antigen binding ability (e.g., Fab, Fab′, F(ab′)2, and Fv). Such fragments are also well known in the art and are regularly employed both in vitro and in vivo. The term “antibody” also includes chimeric antibodies, humanized antibodies, human antibodies, diabodies, linear antibodies, single chain antibodies, multispecific antibodies (e.g., bispecific antibodies), and any other modified configuration of the immunoglobulin molecule that comprises an antigen recognition site of the required specificity, including amino acid sequence variants of antibodies, glycosylation variants of antibodies, and covalently modified antibodies. Immunoglobulin Y (IgY, Y=yolk) refers to antibodies that are produced in the body of avian and can be extracted from egg yolk.

As used herein, the term “viral particle” can mean the fully or partially assembled capsid of a virus that may include an empty particle, a full particle or a sub-particle.

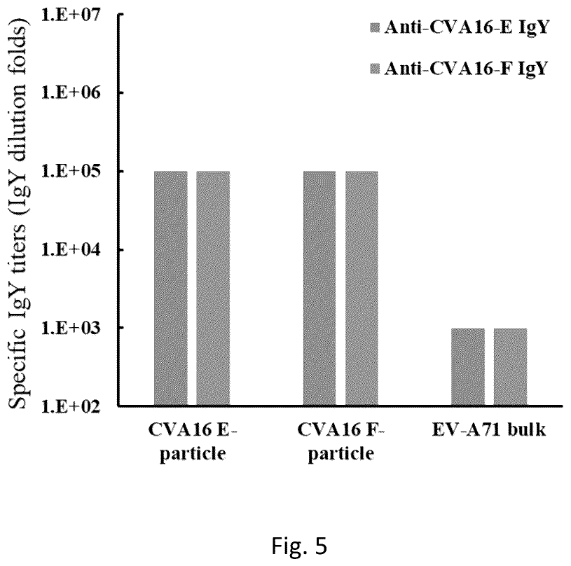

As used herein, the term “full particle” or “F-particle” is a virus particle containing viral genomic nucleic acids encapsulated within a protein shell (capsid). In particular, CVA 16 full particles include CVA16 genomic RNA encapsulated within the protein shell comprised of capsid proteins including viral capsid protein 1 (VP1), Viral capsid protein 2 (VP2), Viral capsid protein 3 (VP3) and Viral capsid protein 4 (VP4). More details are described in 1981 Putnak & Phillips: FIG. 5 & Table 3.

As used herein, the term “empty particle” or “empty capsid” or “E-particle” is a virus particle comprising at least one capsid protein but lack the viral genome. In some embodiments, CVA 16 empty particles include VP0, VP1 and VP3 (without CVA16 genomic RNA). More details are described in 1981 Putnak & Phillips: Fig5 & Table 3.

As used herein, the term “purifying”, “purification”, “isolate”, “isolating”, or “isolation” as used herein, may refer to increasing the degree of purity of a target product (e.g. viral particles) from a sample comprising the target product and one or more impurities. Typically, the degree of purity of target viral particles is increased by removing (completely or partially) at least one impurity from the sample. In some embodiments, viral particles as described herein can be said to be “purified” if it is substantially free of cellular materials or viral components other than the target viral particles. In some cases, purified viral particles can include a preparation containing the viral particles having less than 50%, 40%, 30%, 20% or 10% (by weight) of cellular proteins and other viral components.

The term “immunization” as used herein refers to a process known in the art for inducing an immune response in an animal by introducing an antigenic agent or substance into the animal (e.g., by injection, by mucosal challenge, etc.), which preferably results in a specific immune response (e.g. generating specific antibodies) to the antigenic agent or substance. The antigenic agent or substance can be introduced to the animal, with or without the use of adjuvants.

As used herein, the term “adjuvant” refers to a substance added to an immunogenic composition comprising an antigenic agent that while not having any specific antigenic effect in itself, can stimulate the immune system and increase the immune response to the antigenic agent. Examples of adjuvants include, but are not limited to, alum-precipitate, Freund's complete adjuvant, Freund's incomplete adjuvant, monophosphoryl-lipid A/trehalose dicorynomycolate adjuvant, water in oil emulsion containing Corynebacterium parvum and tRNA, and other substances that accomplish the task of increasing immune response.

As used herein, the term “treatment” refers to the application or administration of one or more active agents to a subject afflicted with a disorder, a symptom or condition of the disorder, or a progression of the disorder, with the purpose to cure, heal, alleviate, relieve, alter, remedy, ameliorate, improve, or affect the disorder, the symptom or condition of the disorder, the disabilities induced by the disorder, or the progression of the disorder.

As used herein, the term “preventing” refers to the preventive measures for a disease or the symptoms or conditions of a disease. The preventive measures include, but are not limited to applying or administering one or more active agents to a subject who has not yet been diagnosed as a patient suffering from the disease or the symptoms or conditions of the disease but may be susceptible or prone to the disease. The purpose of the preventive measures is to avoid, prevent, or postpone the occurrence of the disease or the symptoms or conditions of the disease.

As used herein, the terms “subject,” “individual” and “patient” refer to any mammalian subject for whom diagnosis, prognosis, treatment, or therapy is desired, particularly humans. Other subjects may include cattle, dogs, cats, guinea pigs, rabbits, rats, mice, horses, and so on.

As used herein, the term “diagnosis” as used herein generally includes determination as to whether a subject is likely affected by a given disease, disorder or dysfunction. The skilled persons often make a diagnosis on the basis of one or more diagnostic indicators, i.e., a marker, the presence, absence, or amount of which is indicative of the presence or absence of the disease, disorder or dysfunction. It will be understood in the art that diagnosis does not mean determining the presence or absence of a particular disease with 100% accuracy, but rather an increased likelihood of the presence of certain disease in a subject.

The present invention provides a method for preparing an IgY against CVA16 by immunizing an egg-laying avian with CVA16 particles including CVA16 empty (E) particles and/or CVA16 full (F) particles. Specifically, the immunization is performed with purified CVA16 E particles and/or VA16 F particles. According to the present invention, the immunization is not performed with an infected cell lysate.

Viral particles can be produced by methods known in the art. In general, the production of viral particles requires suitable host cells, including for example, human-derived cell lines, such as human embryonic kidney cell (HEK) 293 cells or rhabdomyosarcoma (RD) cells, mammalian cell lines such as Vero, or insect-derived cell lines such as SF-9 in the case of baculovirus production systems; and suitable media including for example, Modified Eagle Medium (MEM), Dulbecco's Modified Eagle Medium (DMEM), and VP-SFM media. Particularly, suitable host cells are contacted with virus which results in viral infection in the cells; the infected cells are cultured for a period of time sufficient to allow for virus propagation and production of the vial particles; and then the viral particles thus produced are collected. More particularly, before infection, cells are cultured to reach a density of about 10⁵ to 10⁶ cells per mL and then the cells are infected with the virus at a MOI (multiplicity of infection) of about 10⁻² to 10⁻⁵ and cultivated for about 3 to 10 days. The virus particles are then harvested from the culture supernatants, which are subjected to a subsequent procedure, such as concentration, purification and/or inactivation.

In some embodiments, the purification is conducted by liquid chromatography purification, sucrose gradient ultracentrifuge purification, or a combination thereof. Preferably, the purification is conducted by sucrose gradient ultracentrifuge purification. More preferably, 10 to 60% sucrose density gradient is used in the sucrose gradient ultracentrifuge purification.

In some embodiments, the purification is conducted to obtain a fraction of CVA16 empty (E) particles. In particular, a fraction of CVA16 empty (E) particles can be obtained from 19-27% sucrose gradient.

In some embodiments, the purification is conducted to obtain a fraction of CVA16 full (F) particles. In particular, a fraction of CVA16 full (F) particles can be obtained from 29-40% sucrose gradient.

In some embodiments, CVA16 E-particles as described herein has a molecular weight of about 5,000-7,000 kDa. In some particular embodiments, CVA16 E-particles as described herein has a molecular weight of about 5,460-6,060 kDa. In some embodiments, CVA16 E-particles as described herein include VP0 (35-38 kDa), VP1 (32-35 kDa), and VP3 (24-28 kDa).

In some embodiments, CVA16 F-particles as described herein has a molecular weight of about 7,500-8,500 kDa. In some particular embodiments, CVA16 F-particles as described herein has a molecular weight of about 7,836-8,436 kDa. In some embodiments, CVA16 F-particles as described herein include VP1 (32-35 kDa), VP2 (24-28 kDa), VP3 (24-28 kDa), VP4 (6-8 kDa) and viral RNA (RNA: 2,376 kDa).

In some embodiments, CVA16 E-particles and CVA16 F-particles as described herein can be obtained from CVA16 strains as known in the art. One certain example is CVA16 5079 (clinical isolate).

In some embodiments, CVA16 VP0 as described herein is from CVA16 5079 capsid protein VP0 having the amino acid sequence as set forth in SEQ ID NO: 28, or from other CVA16 strains having 80% or more identical, e.g., 85%, 86%, 87%, 88%, 89%, 90%, 91%, 92%, 93%, 94%, 95%, 96%, 97%, 98%, 99%, 99.5% to SEQ ID NO: 28.

In some embodiments, CVA16 VP1 as described herein is from CVA16 5079 capsid protein VP1 having the amino acid sequence as set forth in SEQ ID NO: 29, or from other CVA16 strains having 80% or more identical, e.g., 85%, 86%, 87%, 88%, 89%, 90%, 91%, 92%, 93%, 94%, 95%, 96%, 97%, 98%, 99%, 99.5% to SEQ ID NO: 29.

In some embodiments, CVA16 VP2 as described herein is from CVA16 5079 capsid protein VP2 having the amino acid sequence as set forth in SEQ ID NO: 30, or from other CVA16 strains having 80% or more identical, e.g., 85%, 86%, 87%, 88%, 89%, 90%, 91%, 92%, 93%, 94%, 95%, 96%, 97%, 98%, 99%, 99.5% to SEQ ID NO: 30.

In some embodiments, CVA16 VP3 as described herein is from CVA16 5079 capsid protein VP3 having the amino acid sequence as set forth in SEQ ID NO: 31, or from other CVA16 strains having 80% or more identical, e.g., 85%, 86%, 87%, 88%, 89%, 90%, 91%, 92%, 93%, 94%, 95%, 96%, 97%, 98%, 99%, 99.5% to SEQ ID NO: 31

In some embodiments, CVA16 VP4 as described herein is from CVA16 5079 capsid protein VP4 having the amino acid sequence as set forth in SEQ ID NO: 32, or from other CVA16 strains having 80% or more identical, e.g., 85%, 86%, 87%, 88%, 89%, 90%, 91%, 92%, 93%, 94%, 95%, 96%, 97%, 98%, 99%, 99.5% to SEQ ID NO: 32

In particular, the collected viral particles are inactivated, for example, by formalin treatment. In certain examples, the treatment with formaldehyde is at about 20-45° C. for 2 to 20 days.

To prepare an IgY as described in the present invention, CVA16 viral particles can be mixed with a proper adjuvant as known in this art (e.g. a complete Freund adjuvant) and then administered to a fowl via muscle injection, for example, for immunization. Further immunization of CVA16 viral particles with an incomplete Freund adjuvant, for example, is preferred to boost the immune response in the fowl. In general, one to three times of boost or more is suggested, and the period of time between each immunization is about 7 to 28 days. After the immunization, eggs laid by the fowl are collected and the yolk is isolated from the eggs. In some embodiments, the yolk is further processed as needed e.g. lyophilized for the purpose of easy storage. In some embodiments, the yolk is processed to perform immunoglobulin extraction from the yolk. Many prior art methods of immunoglobulin isolation are available involving use of precipitating agents, such as ethanol, polyethylene glycol, lyotropic salts such as copper sulphate, ammonium sulphate and ammonium phosphate, and caprylic acid. The presence and activity of specific antibodies in the yolk can be confirmed by any method such as an immunological assay known in art e.g. Enzyme-linked immunosorbent assay (ELISA), a method using agglutination reaction and/or a neutralizing test.

In some embodiments, the anti-CVA16 antibody thus prepared specifically recognizes various epitopes of CVA16 capsid proteins. See Table 2.

In some embodiments, the immunization is performed with CVA16 E particles and the anti-CVA16 IgY thus prepared neutralizes CVA16 and EV-A71.

In some embodiments, the immunization is performed with CVA16 F particles and the anti-CVA16 IgY thus prepared neutralizes CVA16 and fail to neutralize EV-A71.

According to the present invention, the anti-CVA16 antibody may be formulated with a carrier to form a composition for purpose of delivery and absorption.

As used herein, a carrier may be a pharmaceutically acceptable carrier. As used herein, “pharmaceutically acceptable” means that the carrier is compatible with an active ingredient in the composition, and preferably can stabilize said active ingredient and is safe to the receiving individual. Said carrier may be a diluent, excipient, matrix or vehicle to the active ingredient. Typically, a composition comprising an antibody as described herein as an active ingredient can be in a form of a solution such as an aqueous solution e.g. a saline solution or it can be provided in powder form. Appropriate excipients also include lactose, sucrose, dextrose, sorbose, mannose, starch, Arabic gum, calcium phosphate, alginates, tragacanth gum, gelatin, calcium silicate, microcrystalline cellulose, polyvinyl pyrrolidone, cellulose, sterilized water, syrup, and methylcellulose. The composition may further contain pharmaceutically acceptable auxiliary substances as required to approximate physiological conditions, for example, pH adjusting and buffering agents, such as sodium acetate, sodium chloride, potassium chloride, calcium chloride, sodium lactate and the like. The form of the composition may be tablets, pills, powder, lozenges, packets, troches, elixers, suspensions, lotions, solutions, syrups, soft and hard gelatin capsules, suppositories, sterilized injection fluid, and packaged powder. The composition of the present invention may be delivered via any physiologically acceptable route, such as oral, parenteral (such as intramuscular, intravenous, subcutaneous, and intraperitoneal), transdermal, suppository, and intranasal methods. In certain embodiments, the composition of the present invention is administered as a liquid injectable formulation which can be provided as a ready-to-use dosage form or as a reconstitutable stable powder.

The anti-CVA16 antibody of the present invention exhibits neutralizing capacity against CAV16 and/or EV-A71 infection. Therefore, the present invention provides a method for treating or preventing CAV16 and/or EV-A71 infection by administering to a subject in need thereof an effective amount of an anti-CVA16 IgY as described herein.

The anti-CVA16 antibody of the present invention is also useful to diagnosis purposes. Therefore, the present invention provides a method for diagnosing CAV16 and/or EV-A71 infection. In particular, the method comprises (i) providing a sample derived from a subject in need thereof; (ii) reacting the sample with an anti-CVA16 IgY as described herein; and (iii) detecting an antigen-antibody reaction between the anti-CVA16 IgY and CAV16 and/or EV-A71 present in the sample.

The present invention is further illustrated by the following examples, which are provided for the purpose of demonstration rather than limitation. Those of skill in the art should, in light of the present disclosure, appreciate that many changes can be made in the specific embodiments which are disclosed and still obtain a like or similar result without departing from the spirit and scope of the invention.

### EXAMPLES

During the propagation of enterovirus in mammalian cells, many types of virus particles are produced. Among them, two major kinds of viral particles are usually harvested the empty (E)-particle (immature virion) and the full (F)-particle (mature virion), which present different immunogenicity in animal study. [Putnak and Phillips, 1981; Liu et al., 2011; Chong et al., 2012; Liu et al., 2016; Lien et al., 2023]. In this study, we immunized laying hens with purified E- and F-CVA16 viral particles, and then purified the CVA16-specific IgYs from egg yolks. We characterize these CVA16-specific IgYs by liner epitope mapping and in silico simulation. The results provide important information that differs from previous studies of mammalian models, and these CVA16-specific IgYs can be used as diagnostic tools and antiviral reagent for therapeutic application to detect and prevent CVA16 and Enterovirus A71 infection.

### 1. Material and Methods

### 1.1. Cells, Media and Viruses

Vero (CCL-81) cells were obtained from the American Type Culture Collection (ATCC, USA). Vero cells were grown in a VP-SFM medium (Gibco). The human rhabdomyosarcoma cell line (RD) was obtained from the Bioresource Collection and Research Center (BCRC), Hsinchu, Taiwan. RD cells were grown in a DMEM medium (Gibco) supplemented with 10% fetal bovine serum (FBS, Hyclone). All cell lines were passaged twice weekly in T-flasks. The CVA16 5079 strain was obtained from National Cheng Kung University Hospital, Taiwan. The genomic sequence of CVA16 5079 was reported in GenBank: AF177911.1 (J Med Virol. 2001 October; 65 (2): 331-9; PLOS One. 2012 Nov. 30; 7 (11): e49973). The EV-A71 E59 strain was obtained from the Taiwan CDC. These virus stocks were prepared from the supernatants of infected RD cells at 3 days post-infection (DPI). The titers of virus were determined by the median tissue culture infectious dose (TCID₅₀) assay.

### 1.2 CVA16 Viral Particle Preparation

Vero cells were grown using VP-SFM in 850 cm² roller bottle for CVA16 propagation. The medium was replaced with 333 mL fresh medium and the cells were infected with CVA16 at the multiplicity of infection (MOI)=10⁻⁴. After 8-day culture, CVA16 virus solution was harvested and concentrated. Two types of CVA16 viral particles (E-particle and F-particle) were purified using sucrose gradient ultracentrifugation according to previous study [Chong et al., 2012]. Specifically, the virus culture supernatant was harvested from the T-flask culture. The cell debris was removed by passage through a 0.65 μm filter (Sartorius, Germany), and the supernatant was concentrated 20-fold with a 100K TFF capsule (Pall). The crude virus concentrate (˜50 mL) was loaded onto a 10-60% continuous sucrose gradient and centrifuged at 32,000 rpm for three hours using a zonal rotor in a Hitachi CP80 ultracentrifuge. The fractions (50 mL per fraction) at 10 to 60% sucrose were collected and individually dialyzed against three exchanges of 1 L PBS at pH 7.4 (Gibco/Life Technologies, Taipei, Taiwan), then stored at 4° C. The fractions were also subjected to SDS-PAGE and Western blot analyses. The fractions identified to contain virus were pooled and concentrated by diafiltration using an Amicon 100K tube (Millipore, Belerica, MA USA) and centrifuged at 3,000×g, then stored at 4° C. The total protein concentration of the purified virus fractions was determined by a BCA protein assay. CVA16 empty (E) particles was identified at 19-27% sucrose gradient, and CVA16 full (F) particles was identified at 29-40% sucrose gradient. The resultant CVA16 viral particles were inactivated by 1/4000 (v/v) formalin at 37° C. for 3 days and stored at 4° C. EV-A71 bulk sample (S05), which composed of mix-particles, was used as control and produced by the PIC/S cGMP facility of the National Health Research Institutes, Taiwan.

1.3 Immunization of Laying Hens with CVA16 and EV-A71 Viral Particles

Each type of CVA16 viral particles were prepared in 0.5 mL of normal saline solution and then emulsified with 0.5 mL of Freund's complete adjuvant (Sigma-Aldrich). In CVA16 E-particle group, 2 chickens (A16-E1 and A16-E2) were immunized with 70 μg/dose antigens; in CVA16 F-particle group, 2 chickens (A16-F1 and A16-F2) were immunized with 40 μg/dose antigens; in EV-A71 bulk group, 2 chickens (EV-A71-B1 and EV-A71-B2) were immunized with 25 μg/dose antigens. The resultant emulsified samples with complete Freund's adjuvant (V/V=0.5 mL+0.5 mL) were used for immunizing the laying hens via intramuscular injection into 5 different sites on the muscles of the thorax. Four weeks after the 1st immunization, the same antigen proteins formulated in incomplete Freund's adjuvant (V/V=0.5 mL+0.5 mL) were used for the 2nd immunization of these hens using the same procedures. Four weeks after the final injection, eggs were harvested and stored at 4° C.

### 1.4 the Extraction of Anti-CVA16 and Anti-EV-A71 IgYs

The extraction of IgYs was followed by previous report for extracting IgY from egg yolks [Yeh et al., 2022]. The method for extracting IgY from egg yolks is conducted as follows. Briefly, the yolk sample with the buffer solution was diluted with the acetate-based buffer solution with a pH ranging from 4.6 to 5.4. The mixture was then stirred for at least 30 min at 4° C., followed by centrifugation. After centrifugation of the mixture at 14,000×g for 30 min, the supernatant was collected. Thereafter, the inorganic ammonium sulfate salt solution with a concentration ranging from 0.05 M to 0.15 M and a saturation degree from 30% to 60% was added into the supernatant to salt out the IgYs. After another stirring for 30 min, the mixture was centrifuged again at 14,000×g for 30 min to precipitate the IgYs. The whole extraction process requires approximately 2 hours.

### 1.5 SDS-PAGE and Western Blotting Analysis

Western blotting analyses were performed using 10% Tris-Glycine SDS-PAGE. Samples were separated by SDS-PAGE and transferred onto a PVDF membrane (Invitrogen) using a Mini Trans-Blot Cell (Bio-Rad) according to the manufacturer's instructions. Primary antibodies such as anti-CV-A16 IgYs, anti-EV-A71 IgY, and previously described enterovirus-related commercial antibodies, were used to detect the enterovirus lysates. Secondary antibodies such as goat anti-rabbit IgG (AP132P, Millipore) conjugated with horseradish peroxidase (HRP), goat anti-mouse IgG HRP (GTX213111-01, GeneTex), and rabbit anti-chicken IgY HRP (A9046, Sigma Aldrich) were then applied to the membrane. Immobilon crescendo western HRP substrate WBLUR0500 (Millipore) was used for chemiluminescence development and the image was captured by the Amersham Imager 600 system (GE Healthcare).

### 1.6 Virus Neutralizing Test

The virus neutralization test (Nt) was performed as described previously [Liu et al., 2011]. Each sample was then serially diluted (2-fold) with culture medium. Two hundred μL of virus solution with titer equal to 200 TCID₅₀ were added to tubes containing 200 μL of the diluted sera. After incubation at 4° C. for 18-24 hours, these samples (100 μL/well) were added to 96-well plates containing RD cells. The cultures were incubated for 6 days at 37° C., and TCID₅₀ were measured after quantifying the CPE in the infected RD cells. The Nt value is the geometric reciprocal of the serum dilution yielding a 50% reduction in the viral titer, was obtained using the Reed-Muench methods.

### 1.7 Specific Titer ELISA Analysis

Three chicken IgYs (Anti-CVA16-E IgY, Anti-CVA16-F IgY, and Anti-EV-A71 IgY) were diluted to recognize the formalin-inactivated viral particles (CVA16 E-particle, CVA16 F-particle, and EV-A71 bulk) by ELISA. Viral particles were coated on a 96-well ELISA plate (Corning) at 10 ng/well in 50 μL coating buffer (1M NaHCO₃, pH9.5) (Liu et al., 2016). After antigen coating, 250 μL of 5% skim milk in PBS was added for blocking. One hundred μL of testing sera and antibodies were added to testing wells and incubated for 2 hours at room temperature. The wells were washed four times with 250 μL wash buffer (PBS+0.05% Tween20), and 100 μL of an HRP-conjugated secondary antibody (1:30,000 dilution; Jackson ImmunoResearch) was added to each well for 30 minutes incubation at room temperature. The plate was washed six times with wash buffer and dried with filter paper. 50 μL of TMB peroxidase substrate (SureBlueTM, KPL) was added for 30 minutes reaction and the reaction was stopped by adding 50 μL of 2N H₂SO₄. The absorbance at 450 nm was measured by an ELISA reader (Spectra Max M2 model, USA).

### 1.8 Peptide-ELISA Analysis

A panel of 165 overlapping synthetic peptides were synthesized using Fmoc chemistry by Kelowna International Scientific Inc. (Taipei Hsien, Taiwan) according to the sequence of VP1 (57×15-mer peptides), VP2 (49×15-mer peptides), VP3 (47×15-mer peptides), and VP4 (12×15-mer peptides) capsid proteins of CVA16 [Chong et al, 2012]. Each peptide contained 15 amino acids, 10 residues of which overlapped with the adjacent peptides. The reactivity of the antibody to each synthetic peptide was analyzed by an enzyme-linked immunosorbent assay (ELISA) according to the protocol previously reported by Chong et al. [Chong et al, 2012]. Peptides and viral particles were coated on a 96-well ELISA plate (Corning) at 0.5 μg/well in 50 μL coating buffer (0.1 M NaHCO₃, pH9.5) [Liu et al., 2016]. After antigen coating, 250 μL of 5% skim milk in PBS was added for blocking. Fifty μL of primary antibodies were added to testing wells and incubated for 2 hours at room temperature. The wells were washed four times with 250 μL wash buffer (PBS+0.05% Tween20), and 100 μL of an HRP-conjugated secondary antibody that includes the goat anti-mouse IgG HRP (1:5,000 dilution; GeneTex), the goat anti-rabbit IgG HRP (1:4,000 dilution; Millipore), and anti-chicken IgY HRP (1:20,000 dilution; Sigma Aldrich) were added to each well for 1 hour incubation at room temperature. The plate was washed six times with wash buffer and dried with filter paper. 50 μL of TMB peroxidase substrate (eBioscience) was added for 30 minutes reaction and the reaction was stopped by adding 50 μL of 2N H₂SO₄. The absorbance at 450 nm was measured by an ELISA reader (Spectra Max M2 model, USA).

### 1.9 Sequence Alignment and Structure Homology Modeling Prediction

The genome sequences of all related CVA16 strains were obtained from the NCBI PubMed website (http://www.ncbi.nlm.nih.gov/pubmed/). The genome sequences of the CVA16 P1 gene were aligned using the ClustalW2 program (http://www.ebi.ac.uk/Tools/clustalw2/index.html). The three-dimensional structure of CVA16 (PDB: 6LHA) was used to predict the possible epitope position by homology modeling [He et al., 2020]. The Cn3D v4.3 graphics (http://www.ncbi.nlm.nih.gov) were used to display the position of the identified peptides.

### 1.10 Ethics Statement

The animal experiment was conducted in accordance with the guidelines of the Laboratory Animal Center of the National Defense Medical Center (NDMC), Taiwan. The animal use protocols have been reviewed and approved by the NDMC Institutional Animal Care and Use Committee (NDMC-IACUC approval No.: AN-110-16 & AN-111-26).

### 2. Results

### 2.1 Preparation of E- and F-CVA16 Viral Particles

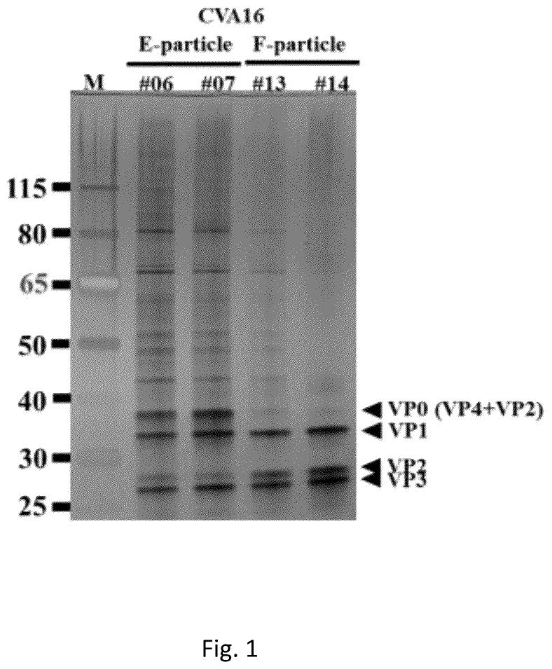

To prepare two types of CVA16 viral particles, the concentrated CVA16 solution were purified using sucrose gradient ultracentrifugation. As shown in FIG. 1, fractions #06 and #07 (19-27% sucrose gradient) were collected as CVA16 E-particle, while fractions #13 and #14 (29-40% sucrose gradient) were collected as CVA16 F-particle. The major viral proteins of CVA16 E-particle are VP0, VP1, and VP3. The major viral proteins of CVA16 F-particle are VP1, VP2, VP3, and VP4.

### 2.2 Production and Characterization of Anti-CVA16 IgYs and Anti-EV-A71 IgY

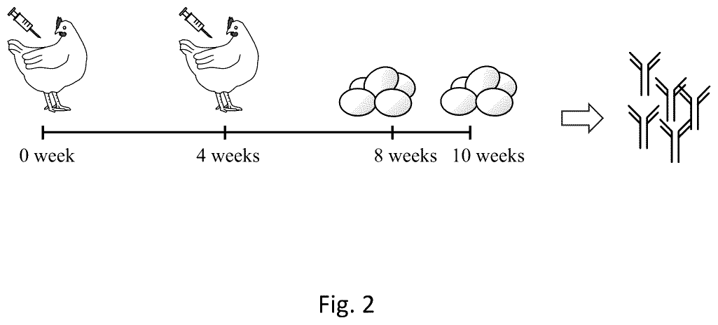

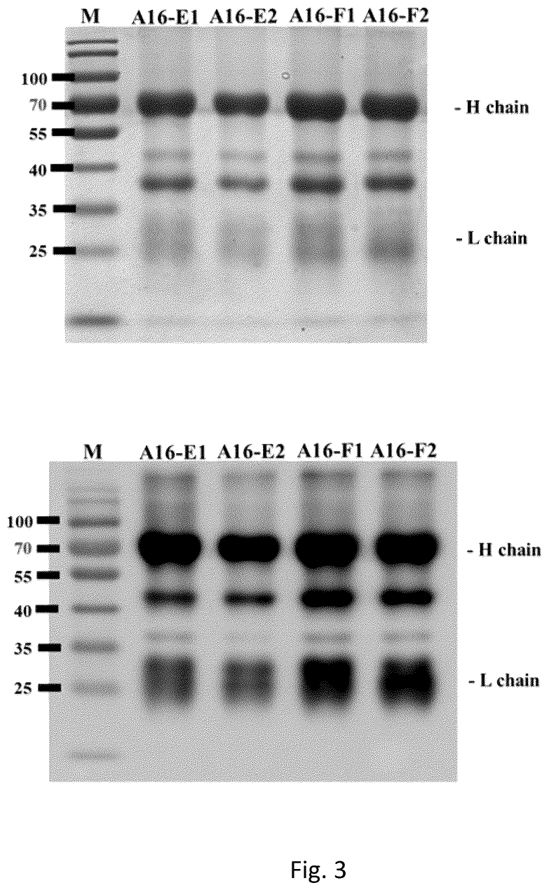

To generate anti-CVA16 IgYs and anti-EV-A71 IgY, chickens were immunized with two types of CVA16 viral particles and EV-A71 bulk, respectively. The immunization flow chart was shown in FIG. 2. After purification of the immunized yolks, the protein profiles of the anti-CVA16 IgYs (anti-CVA16-E IgY and anti-CVA16-F IgY) were identified using SDS-PAGE and western blot (FIG. 3). 2.3 Antigen recognition of anti-CVA16 IgYs and anti-EV-A71 IgY

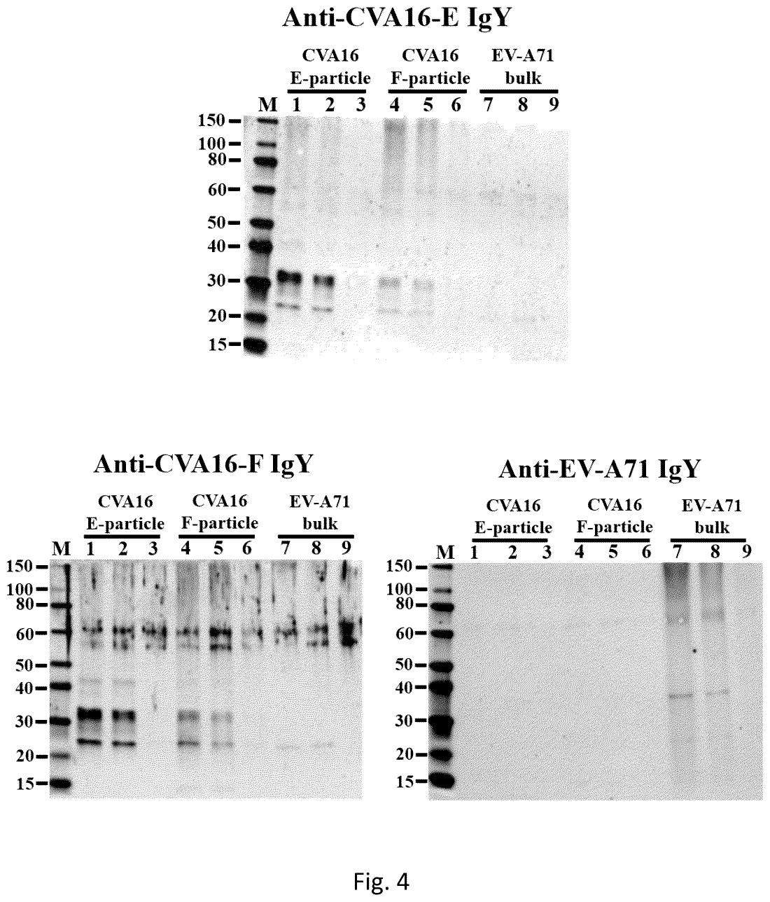

To evaluate the antigen recognition of anti-CVA16 IgYs and anti-EV-A71 IgY, three types of antigens (CVA16 E-particle, CVA16 F-particle, and EVA71 bulk) were loaded in SDS-PAGE for western blot assay (FIG. 4). The anti-CVA16-E IgY and anti-CVA16-F IgY recognized both the viral proteins of E- and F-particles of CVA16. Anti-CVA16 IgYs are found to also recognize the viral proteins of EV-A71, although to a lesser extent. The anti-EV-A71 IgY recognized the viral proteins of EV-A71, but did not weak recognize the viral proteins of CVA16. This shows that specific antibodies of anti-CVA16 IgYs were produced to recognized the viral proteins of CVA16, and anti-EV-A71 IgY was specifically recognized the viral proteins of EV-A71.

### 2.4 Reactivity and Specificity of Anti-CVA16 IgYs

In order to confirm the reactivity and specificity of the anti-CVA16 IgYs generated from CVA16 E-particle and F-particle, three enteroviral particles (CVA16 E-particle, CVA16 F-particle, and EVA71 bulk) were tested in this ELISA analysis. For comparison, the anti-CVA16 IgYs diluted to 1×10³ dilutions, which still recognized the EV-A71 particles, is defined as the baseline. The two types of anti-CVA16 IgYs were found to highly recognize CVA16 E-particle and CVA16 F-particle even at 1×10⁵ dilutions. The results show that the anti-CVA16 IgYs have specific reactivity to the CVA16 particles than EV-A71 particles (FIG. 5)

### 2.5 Neutralization of Anti-CVA16 IgYs and Anti-EV-A71 IgY

Our previous study has shown that the E- and F-particles of CVA16 had profound difference in immunogenicity in mice [Chong et al., 2012]. To investigate whether the E- and F-particles of CVA16 also reveal this variance in chicken model, anti-CVA16-E IgY, anti-CVA16-F IgY and anti-EV-A71 IgY were evaluated by the neutralization assay (Nt titer) against CVA16 and EV-A71 infection, respectively. The results were shown in Table 1. The anti-CVA16-F IgY presented higher neutralizing titers against CVA16 (Nt=5145.3±1646.5) than the anti-CVA16-E IgY (Nt=354.8±0). Interesting, the anti-CVA16-E IgY presented cross-neutralizing titers against EV-A71 (Nt=12366.9±1621.7). However, the anti-CVA16-F IgY failed to neutralize the EV-A71 infection (Nt<16). The anti-EV-A71 IgY samples can neutralize EV-A71 (Nt>524288), but that failed to neutralize CVA16 (Nt<16). Our result indicated that the E- and F-particles of CVA16 should be used as the immunogens for the induction of a good and specific neutralization response against CVA16 in chicken model. The anti-CVA16-E IgY can cross-neutralize the EV-A71. In addition, the anti-EV-A71 IgY cannot cross-neutralize the CVA16.

TABLE 1
Neutralization titer of anti-CVA16 IgYs and anti-EV-A71 IgY.

| | Sample | | |
|---|---|---|---|
| Virus | Anti-CVA16-E | Anti-CVA16-F | Anti-EV-A71 |
| CVA16 | 354.8 ± 0 | 5145.3 ± 1646.5 | <16 |
| EV-A71 | 12366.9 ± 1621.7 | <16 | >524288 |

(Nt = Mean ± SD)

### 2.6 Liner Epitope Mapping of Anti-CVA16 IgYs and Anti-EV-A71 IgY

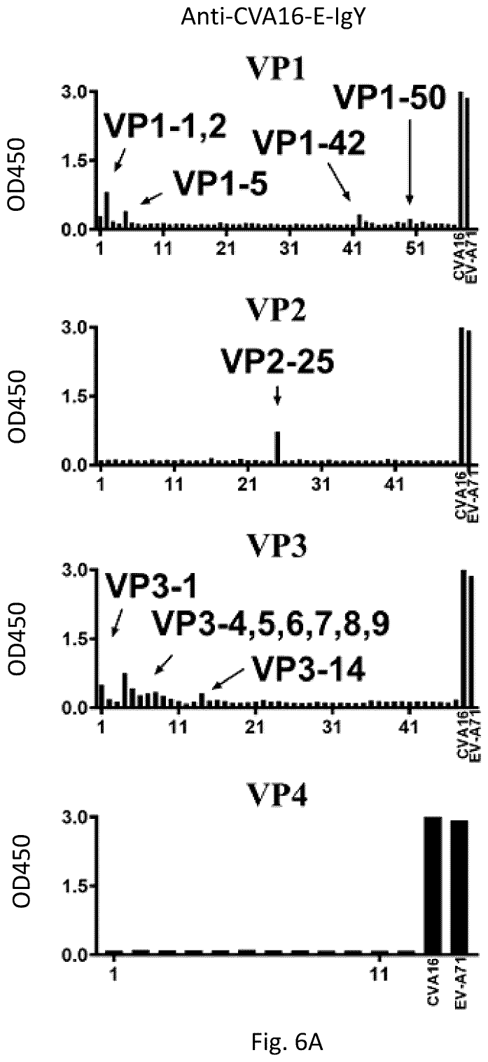

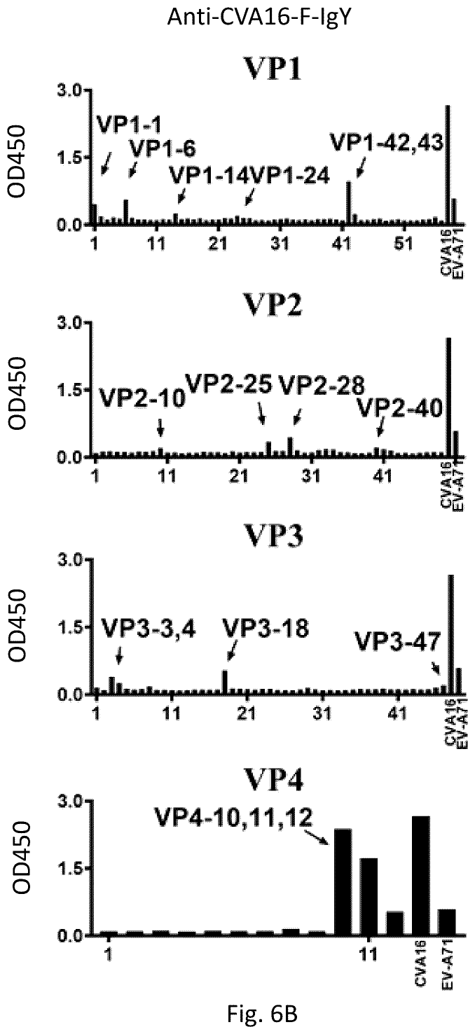

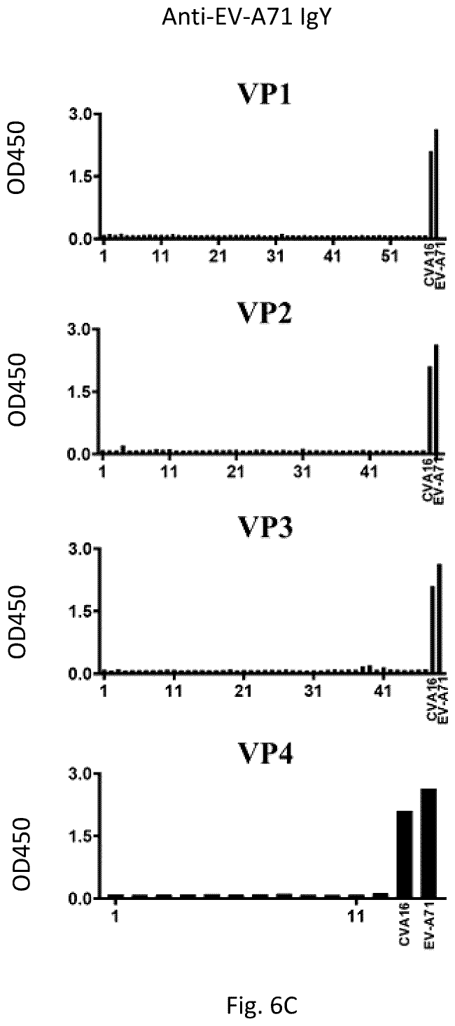

Although current results indicate that both mouse and rabbit antisera have CVA16 virus-specific neutralizing antibodies, it remains worth investigating whether IgY antibodies could cross-react with the reported liner epitopes of CVA16. Our previous study has shown that the antisera of the E- and F-particles of CVA16 induced in mic recognized different liner epitopes [Chong et al., 2012]. As shown in FIG. 4, western blot analyses indicated that the anti-CVA16 IgYs react with two major bands of CVA16. The protein molecular weights of VP0 (36 kDa) and VP1 (33 kDa) are very close, and the protein molecular weights of VP2 (28 kDa) and VP3 (27 kDa) are also very close. The specificity of these anti-CVA16 IgYs were screened by peptide-ELISA for their reactivity with 165 overlapping synthetic peptides (15-mer) that covered all 4 structural proteins (VP1 to VP4) of CVA16. The anti-CVA16-E IgY and anti-CVA16-F IgY reacted with several synthetic peptides of CVA16 in peptide-ELISA studies (OD. ≥0.2), which indicated different spectrum of liner epitope reactivity in CVA16 (FIGS. 6A-6C). The reactivity of these anti-CVA16 IgYs with CVA16 synthetic peptides (SEQ ID NO:1 to 27) were summarized in Table 2. The anti-CVA16-E IgY reacted with the VP1-1 (SEQ ID NO: 1; GDPIADMIDQTVNNQ), VP1-2 (SEQ ID NO: 2; DMIDQTVNNQVNRSL), VP1-5 (SEQ ID NO: 3; TALQVLPTAADTEAS), VP1-42 (SEQ ID NO: 7; DGYPTFGEHLQANDL), VP1-50 (SEQ ID NO: 9; SITLRVYMRIKHVRA), VP2-25 (SEQ ID NO: 11; ALLVAVLPEYVLGTI), VP3-1 (SEQ ID NO: 14; GIPTELKPGTNQFLT), VP3-4 (SEQ ID NO: 16; TDDGVSAPILPGFHP), VP3-5 (SEQ ID NO: 17; SAPILPGFHPTPPIH), VP3-6 (SEQ ID NO: 18; PGFHPTPPIHIPGEV), VP3-7 (SEQ ID NO: 19; TPPIHIPGEVHNLLE), VP3-8 (SEQ ID NO: 20; IPGEVHNLLEICRVE), VP3-9 (SEQ ID NO: 21; HNLLEICRVETILEV), and VP3-14 (SEQ ID NO: 22; MQRLCFPVSVQSKTG) peptides. The anti-CVA16-F IgY reacted with the VP1-1 (SEQ ID NO: 1; GDPIADMIDQTVNNQ), VP1-6 (SEQ ID NO: 4; LPTAADTEASSHRLG), VP1-14 (SEQ ID NO: 5; TRCVLNHHSTQETAI), VP1-24 (SEQ ID NO: 6; YAQLRRKCELFTYMR), VP1-42 (SEQ ID NO: 7; DGYPTFGEHLQANDL), VP1-43 (SEQ ID NO: 8; FGEHLQANDLDYGQC), VP2-10 (SEQ ID NO: 10; DATAVDKPTRPDVSV), VP2-25 (SEQ ID NO: 11; ALLVAVLPEYVLGTI), VP2-28 (SEQ ID NO: 12; AGGTGNENSHPPYAT), VP2-40 (SEQ ID NO: 40; PYMNTVPFDSALNHC), VP3-3 (SEQ ID NO: 15; NQFLTTDDGVSAPIL), VP3-4 (SEQ ID NO: 16; TDDGVSAPILPGFHP), VP3-18 (SEQ ID NO: 23; FRADPGRDGPWQSTI), VP3-47 (SEQ ID NO: 24; DTEDIEQTANIQ), VP4-10 (SEQ ID NO: 25; MSQDPKKFTDPVMDV), VP4-11 (SEQ ID NO: 26; KKFTDPVMDVIHEMA), and VP4-12 (SEQ ID NO: 27; PVMDVIHEMAPPLK) peptides. Anti-EV-A71 IgY generated from EV-A71 viral particles failed to react with specific peptides of CVA16 in peptide-ELISA studies, but reacted with EV-A71 and CVA16 particles.

TABLE 2
Summary of anti-CVA16 IgYs reacted CVA16
synthetic peptides.

| | | | Anti- | Anti- |
|---|---|---|---|---|
| | Amino acid | Peptide | CVA16-E | CVA16-F |
| SEQ ID NO. | sequence | code | IgY | IgY |
| SEQ ID NO: 1 | GDPIADMIDQTVNNQ | VP1-1 | ▪ | ▪ |
| SEQ ID NO: 2 | DMIDQTVNNQVNRSL | VP1-2 | ▪ | |
| SEQ ID NO: 3 | TALQVLPTAADTEAS | VP1-5 | ▪ | |
| SEQ ID NO: 4 | LPTAADTEASSHRLG | VP1-6 | | ▪ |
| SEQ ID NO: 5 | TRCVLNHHSTQETAI | VP1-14 | | ▪ |
| SEQ ID NO: 6 | YAQLRRKCELFTYMR | VP1-24 | | ▪ |
| SEQ ID NO: 7 | DGYPTFGEHLQANDL | VP1-42 | ▪ | ▪ |
| SEQ ID NO: 8 | FGEHLQANDLDYGQC | VP1-43 | | ▪ |
| SEQ ID NO: 9 | SITLRVYMRIKHVRA | VP1-50 | ▪ | |
| SEQ ID NO: 10 | DATAVDKPTRPDVSV | VP2-10 | | ▪ |
| SEQ ID NO: 11 | ALLVAVLPEYVLGTI | VP2-25 | ▪ | ▪ |
| SEQ ID NO: 12 | AGGTGNENSHPPYAT | VP2-28 | | ▪ |
| SEQ ID NO: 13 | PYMNTVPFDSALNHC | VP2-40 | | ▪ |
| SEQ ID NO: 14 | GIPTELKPGTNQFLT | VP3-1 | ▪ | |
| SEQ ID NO: 15 | NQFLTTDDGVSAPIL | VP3-3 | | ▪ |
| SEQ ID NO: 16 | TDDGVSAPILPGFHP | VP3-4 | ▪ | ▪ |
| SEQ ID NO: 17 | SAPILPGFHPTPPIH | VP3-5 | ▪ | |
| SEQ ID NO: 18 | PGFHPTPPIHIPGEV | VP3-6 | ▪ | |
| SEQ ID NO: 19 | TPPIHIPGEVHNLLE | VP3-7 | ▪ | |
| SEQ ID NO: 20 | IPGEVHNLLEICRVE | VP3-8 | ▪ | |
| SEQ ID NO: 21 | HNLLEICRVETILEV | VP3-9 | ▪ | |
| SEQ ID NO: 22 | MQRLCFPVSVQSKTG | VP3-14 | ▪ | |
| SEQ ID NO: 23 | FRADPGRDGPWQSTI | VP3-18 | | ▪ |
| SEQ ID NO: 24 | DTEDIEQTANIQ | VP3-47 | | ▪ |
| SEQ ID NO: 25 | MSQDPKKFTDPVMDV | VP4-10 | | ▪ |
| SEQ ID NO: 26 | KKFTDPVMDVIHEMA | VP4-11 | | ▪ |
| SEQ ID NO: 27 | PVMDVIHEMAPPLK | VP4-12 | | ▪ |

▪: Anti-CVA16 IgY reacted synthetic peptide.

### 2.7 Potential Location of CVA16 Neutralization Epitopes Estimated by in Silico Simulation

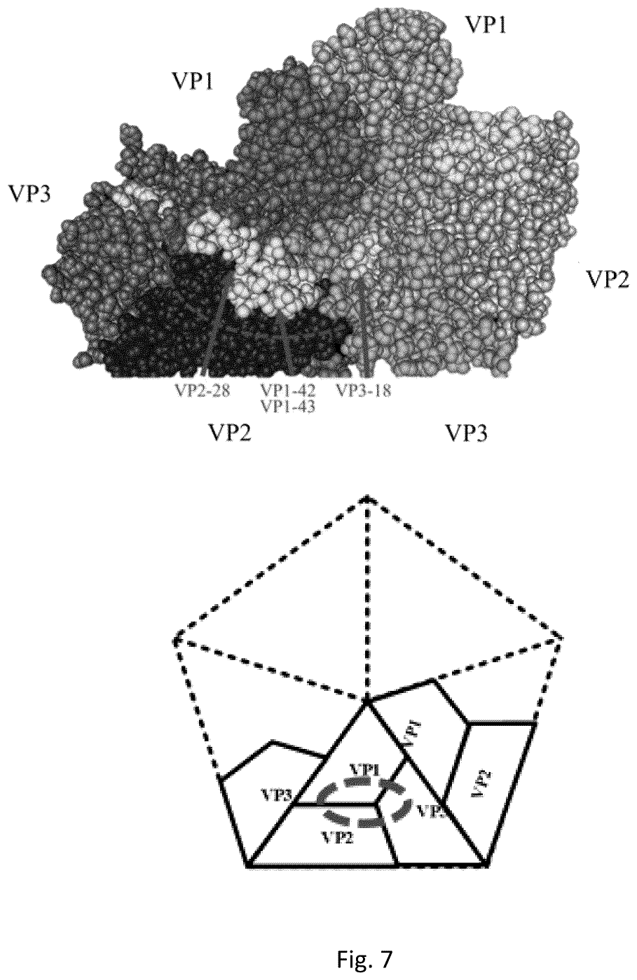

To define the locations and spatial relationship of these liner epitopes identified in this study. The viral structure model of CVA16 190 strain (PDB: 6LHA) was used as the template for the construction of the molecular structure of CVA16. The structure protein sequences (VP1 to VP4) of the CVA16 5079 strain were aligned using the Clustal W2 program. After labeling these liner epitopes of anti-CVA16 IgYs in structure protein sequences, and the results showed that the VP1-42, VP1-43, VP2-28, and VP3-18 peptides form a region in the canyon at the junction site of VP1, VP2, and VP3 within the capsid (FIG. 7). The putative molecular structure of CVA16 illustrates the importance of these neutralization epitopes. In previous studies, the VP1-42, VP1-43, and VP2-28 were reported to be the important epitope sequences [Fang and Liu, 2018]. These results further confirm that the VP3-18 peptide is an important functional cross-neutralizing epitope. In addition, the reacted peptides of the anti-CVA16-F IgY highly fitted this predicted region (VP1-42, VP1-43, VP2-28, and VP3-18 peptides) in the structure. This likely explains why anti-CVA16-F IgY was able to recognize and highly neutralize CVA16 than anti-CVA16-E IgY.

### SEQUENCE INFORMATION

| | | |
|---|---|---|
| Strain | Sequence | Size |

VP0

| | | |
|---|---|---|
| CVA16_ | MGSQVSTORSGSHENSNSASEGSTINYTTINYYKDAY | 323 |
| 5079 | AASAGRQDMSQDPKKFTDPVMDVIHEMAPPLKSPSAE | A.A. |
| | ACGYSDRVAQLTIGNSTITTQEAANIVIAYGEWPEYC | |
| | PDTDATAVDKPTRPDVSVNRFFTLDTKSWAKDSKGWY | |
| | WKFPDVLTEVGVFGQNAQFHYLYRSGFCVHVQCNASK | |
| | FHQGALLVAVLPEYVLGTIAGGTGNENSHPPYATTQP | |
| | GQVGAVLTHPYVLDAGIPLSQLTVCPHQWINLRTNNC | |
| | ATIIVPYMNTVPFDSALNHCNFGLLVVPVVPLDENAG | |
| | ATSEIPITVTIAPMCAEFAGLRQAVKQ (SEQ ID | |
| | NO: 28) | |

VP1

| | |
|---|---|
| GDPIADMIDQTVNNQVNRSLTALQVLPTAADTEASSH | 297 |
| RLGTGVVPALQAAETGASSNASDKNLIETRCVLNHHS | A.A. |
| TQETAIGNFFSRAGLVSIITMPTTGTQNTDGYVNWDI | |
| DLMGYAQLRRKCELFTYMRFDAEFTFVVAKPNGELVP | |
| QLLQYMYVPPGAPKPTSRDSFAWQTATNPSVFVKMTD | |
| PPAQVSVPEMSPASAYQWFYDGYPTFGEHLQANDLDY | |
| GQCPNNMMGTFSIRTVGTEKSPHSITLRVYMRIKHVR | |
| AWIPRPLRNQPYLFKTNPNYKGNDIKCTSTSRDKITT | |
| L (SEQ ID NO: 29) | |

VP2

| | | |
|---|---|---|
| CVA16_ | SPSAEACGYSDRVAQLTIGNSTITTQEAANIVIAYGE | 254 |
| 5079 | WPEYCPDTDATAVDKPTRPDVSVNRFFTLDTKSWAKD | A.A. |
| | SKGWYWKFPDVLTEVGVFGQNAQFHYLYRSGFCVHVQ | |
| | CNASKFHQGALLVAVLPEYVLGTIAGGTGNENSHPPY | |
| | ATTQPGQVGAVLTHPYVLDAGIPLSQLTVCPHQWINL | |
| | RTNNCATIIVPYMNTVPFDSALNHCNFGLLVVPVVPL | |
| | DFNAGATSEIPITVTIAPMCAEFAGLRQAVKQ | |
| | (SEQ ID NO: 30) | |

VP3

| | | |
|---|---|---|
| CVA16_ | GIPTELKPGTNQFLTTDDGVSAPILPGFHPTPPIHIP | 242 |
| 5079 | GEVHNLLEICRVETILEVNNLKTNETTPMQRLCFPVS | A.A. |
| | VQSKTGELCAAFRADPGRDGPWQSTILGOLCRYYTQW | |
| | SGSLEVTFMFAGSFMATGKMLIAYTPPGGNVPADRIT | |
| | AMLGTHVIWDFGLQSSVTLVVPWISNTHYRAHARAGY | |
| | FDYYTTGIITIWYQTNYVVPIGAPTTAYIVALAAAQD | |
| | NFTMKLCKDTEDIEQTANIQ (SEQ ID NO: 31) | |

VP4

| | | |
|---|---|---|
| CVA16_ | MGSQVSTQRSGSHENSNSASEGSTINYTTINYYKDAY | 69 |
| 5079 | AASAGRQDMSQDPKKFTDPVMDVIHEMAPPLK | A.A. |
| | (SEQ ID NO: 32) | |

### REFERENCES

Solomon T, Lewthwaite P, Perera D, Cardosa M J, McMinn P, Ooi M H. 2010. Virology, epidemiology, pathogenesis, and control of enterovirus 71. Lancet Infect Dis 10:778-790.
Klein M, Chong P. Is a multivalent hand, foot, and mouth disease vaccine feasible? Human Vaccines and Immunotherapeutics. 2015 November; 11 (11): 2688-2704.
Fang C Y, Liu C C. Recent development of enterovirus A vaccine candidates for the prevention of hand, foot, and mouth disease. Expert Rev Vaccines 2018; 17:819-831.
Fang C Y, Liu C C. Novel strategies for the development of hand, foot, and mouth disease vaccines and antiviral therapies. Expert Opin Drug Discov 2022; 17:27-39.
Chou A H, Liu C C, Chang J Y, et al. Formalin-inactivated EV71 vaccine candidate induced cross-neutralizing antibody responses against subgenotypes B1, B4, B5 and C4A in adult volunteers. PLOS One. 2013; 8 (11): e79783.
Mao Q, Wang Y, Yao X, et al. Coxsackievirus A16: Epidemiology, diagnosis and vaccine. Hum Vaccin Immunother. 2014; 10 (2): 360-367.
Yuan X, Liu Z, Wan L, Liu W, Huang Y, Cao S. Recent advances in anti-coxsackievirus A16 viral drug research. Future Med Chem. 2023 January; 15 (1): 97-117. doi: 10.4155/fmc-2022-0195.
Aswathyraj S, Arunkumar G, Alidjinou E K, et al. Hand, foot and mouth disease (HFMD): emerging epidemiology and the need for a vaccine strategy. Med Microbiol Immunol. 2016; 205 (5): 397-407.
Tikute S, Lavania M. Hand, Foot, and Mouth Disease (HFMD) in India: A Review on Clinical Manifestations, Molecular Epidemiology, Pathogenesis, and Prevention. Indian Dermatol Online 2023 J. May 25; 14 (4): 475-481. doi: 10.4103/idoj.idoj_423_22.
Li R, Lin C, Dong S, Li J, Liang Z, Yang Y, Huo D, Gao Z, Jia L, Zhang D, Wang X, Wang Q. Phylogenetics and phylogeographic characteristics of coxsackievirus A16 in hand foot and mouth disease and herpangina cases collected in Beijing, China from 2019 to 2021. J Med Virol. 2023 August; 95 (8): e28991. doi: 10.1002/jmv.28991.
Yu W, Xu H, Yin C. Molecular epidemiology of human coxsackievirus A16 strains. Biomed Rep. 2016; 4 (6): 761-764.
Leslie G A and Clem L W. Phylogeny of Immunoglobulin Structure and Function. III. Immunoglobulins of the Chicken. J Exp Med. 1969 Nov. 30; 130 (6): 1337-1352. doi: 10.1084/jem.130.6.1337
Suresh L G, Indhuprakash S T, Gandhi S, Diraviyam T. Amalgamation of nanotechnology with chicken IgY to enrich therapeutic and diagnostic applications: a systematic review. Immunotherapy. 2023 August; 15 (11): 867-884. doi: 10.2217/imt-2022-0304.
Wang H, Zhong Q, Lin J. Egg Yolk Antibody for Passive Immunization: Status, Challenges, and Prospects. J Agric Food Chem. 2023 Apr. 5; 71 (13): 5053-5061. doi: 10.1021/acs.jafc.2c09180.
Yakhkeshi S, Wu R, Chelliappan B, Zhang X. Trends in industrialization and commercialization of IgY technology. Front Immunol. 2022 Oct. 20; 13:991931. doi: 10.3389/fimmu.2022.991931.
Yang Y E, Wen J, Zhao S, Zhang K, Zhou Y. Prophylaxis and therapy of pandemic H1N1 virus infection using egg yolk antibody. J Virol Methods. 2014 September; 206:19-26. doi: 10.1016/j.jviromet.2014.05.016.
Yeh C T, Lee C Y, Ho Y J, Chen S A, Chen L Y, Liu P C, Chin Y F, Chen A Y, Hsieh P S, Hung Y J, Chen C C, Wang Y C, Lee G C. Immunoglobulin Y Specific for SARS-CoV-2 Spike Protein Subunits Effectively Neutralizes SARS-COV-2 Infectivity and Ameliorates Disease Manifestations In Vivo. Biomedicines. 2022 Nov. 1; 10 (11): 2774. doi: 10.3390/biomedicines10112774.
Liou J F, Chang C W, Tailiu J J, Yu C K, Lei H Y, Chen L R, Tai C. Passive protection effect of chicken egg yolk immunoglobulins on enterovirus 71 infected mice. Vaccine. 2010 Nov. 29; 28 (51): 8189-96. doi: 10.1016/j.vaccine.2010.09.089.
Nie W, Zhao C, Guo X, Sun L, Meng T, Liu Y, Song X, Xu K, Wang J, Li J. Preparation and identification of chicken egg yolk immunoglobulins against human enterovirus 71 for diagnosis of hand-foot-and-mouth disease. Anal Biochem. 2019 May 15; 573:44-50. doi: 10.1016/j.ab.2019.02.029.
Gao E, Wu S, Xu Q, Zeng Y, Tan N, He S, Yang Y, Wei J. Enterovirus type 71-immunized chicken egg yolk immunoglobulin has cross antiviral activity against coxsackievirus A16 in vitro. Exp Ther Med. 2019 July; 18 (1): 332-341. doi: 10.3892/etm.2019.7529.
Mwale P F, Lee C H, Huang P N, Tseng S N, Shih S R, Huang H Y, Leu S J, Huang Y J, Chiang L C, Mao Y C, Wang W C, Yang Y Y. In Vitro Characterization of Neutralizing Hen Antibodies to Coxsackievirus A16. Int J Mol Sci. 2021 Apr. 16; 22 (8): 4146. doi: 10.3390/ijms22084146.
Lee C H, Huang P N, Mwale P F, Wang W C, Leu S J, Tseng S N, Shih S R, Chiang L C, Mao Y C, Tsai B Y, Dlamini N B, Nguyen T C, Tsai C H, Yang Y Y. The Bottlenecks of Preparing Virus Particles by Size Exclusion for Antibody Generation. Int J Mol Sci. 2022 Oct. 26; 23 (21): 12967. doi: 10.3390/ijms232112967.
Putnak J R, Phillips B A. Picornaviral structure and assembly, Microbiol Rev. 45 (1981) 287-315. https://doi.org/10.1128/mr.45.2.287-315.1981.
Liu C C, Guo M S, Lin F H, Hsiao K N, Chang K H, Chou A H, Wang Y C, Chen Y C, Yang C S, Chong P C. 2011a. Purification and characterization of enterovirus 71 viral particles produced from vero cells grown in a serum-free microcarrier bioreactor system. PLOS One 6: e20005.
Chong P, Guo M S, Lin F H, Hsiao K N, Weng S Y, Chou A H, Wang J R, Hsieh S Y, Su I J, Liu C C. Immunological and biochemical characterization of coxsackie virus A16 viral particles. PLOS One. 2012; 7 (11): e49973. doi: 10.1371/journal.pone.0049973.
Liu C C, Guo M S, Wu S R, Lin H Y, Yang Y T, Liu W C, Chow Y H, Shieh D B, Wang J R, Chong P. Immunological and biochemical characterizations of coxsackievirus A6 and A10 viral particles. Antiviral Res. 2016 May; 129:58-66. doi: 10.1016/j.antiviral.2016.02.008.
Lien S C, Shen Y S, Lin H Y, Wu S R, Fang C Y, Chen C H, Chen Y A, Chong P C, Huang M H, Chow Y H, Wang J R, Wu S C, Liu C C. Propagation and immunological characterization of coxsackievirus A10 in a serum-free HEK293A cell culture system. Virus Res. 2023 May; 329:199101. doi: 10.1016/j.virusres.2023.199101.
He M, Xu L, Zheng Q, Zhu R, Yin Z, Zha Z, Lin Y, Yang L, Huang Y, Ye X, Li S, Hou W, Wu Y, Han J, Liu D, Li Z, Chen Z, Yu H, Que Y, Wang Y, Yan X, Zhang J, Gu Y, Zhou Z H, Cheng T, Li S, Xia N. Identification of Antibodies with Non-overlapping Neutralization Sites that Target Coxsackievirus A16. Cell Host Microbe. 2020 Feb. 12; 27 (2): 249-261.e5. doi: 10.1016/j.chom.2020.01.003.
Leiva C L, Gallardo M J, Casanova N, Terzolo H, Chacana P. IgY-technology (egg yolk antibodies) in human medicine: A review of patents and clinical trials. Int Immunopharmacol. 2020 April; 81:106269. doi: 10.1016/j.intimp.2020.106269.
Yeh C T, Lee C Y, Ho Y J, Chen S A, Chen L Y, Liu P C, Chin Y F, Chen A Y, Hsieh P S, Hung Y J, Chen C C, Wang Y C, Lee G C. Immunoglobulin Y Specific for SARS-CoV-2 Spike Protein Subunits Effectively Neutralizes SARS-COV-2 Infectivity and Ameliorates Disease Manifestations In Vivo. Biomedicines. 2022 Nov. 1; 10(11): 2774.
Yan J J, Su I J, Chen P F, Liu C C, Yu C K, Wang J R. Complete genome analysis of enterovirus 71 isolated from an outbreak in Taiwan and rapid identification of enterovirus 71 and coxsackievirus A16 by RT-PCR. J Med Virol. 2001 October; 65(2): 331-9. doi: 10.1002/jmv.2038.

1. A method for preparing a yolk immunoglobulin (IgY) against coxsackievirus A6 (CVA16) and/or enterovirus A71 (EV-A71) comprising
(i) immunizing an egg-laying avian with CVA16 particles selected from the group consisting of CVA16 empty (E) particles, CVA16 full (F) particles and a combination thereof to obtain an immunized egg product from the avian; and optionally
(ii) extracting and purifying an anti-CVA16 IgY from the immunized egg product.

2. The method of claim 1, wherein the CVA16 particles are purified.

3. The method of claim 1, wherein the CVA16 particles are CVA 16 E particles.

4. The method of claim 1, wherein the CVA16 particles are CVA16 F particles.

5. The method of claim 1, wherein the CVA16 particles are CVA16 E particles and CVA16 F particles.

6. The method of claim 1, wherein the egg-laying avian is selected from the group consisting of chicken, turkey, goose, duck, pheasant, quail, pigeon and ostrich.

7. The method of claim 6, wherein the egg-laying avian is chicken.

8. The method of claim 1, wherein the CVA16 particles are formulated with an adjuvant.

9. The method of claim 1, wherein the immunizing of the egg-laying avian comprises administering the egg-laying avian with a first injection, followed by one or more booster injections, of the CVA16 particles.

10. The method of claim 1, wherein the CVA16 particles are CVA16 E particles and the anti-CVA16 IgY thus prepared neutralizes CVA16 and enterovirus A71 (EV-A71).

11. The method of claim 1, wherein the CVA16 particles are CVA16 F particles and the anti-CVA16 IgY thus prepared neutralizes CVA16 and does not neutralize enterovirus A71 (EV-A71).

12. An anti-CVA16 IgY prepared by the method of claim 1.

13. The anti-CVA16 IgY of claim 12, wherein the IgY specifically binds to
(i) an epitope of capsid protein VP1 selected from the group consisting of VP1-1, VP1-2, VP1-5, VP1-6, VP1-14, VP-24, VP1-42, VP1-43, VP1-50 and any combination thereof, as shown in Table 2;
(ii) an epitope of capsid protein VP2 selected from the group consisting of VP2-10, VP2-25, VP2-28, VP2-40 and any combination thereof, as shown in Table 2;
(iii) an epitope of capsid protein VP3 selected from the group consisting of VP3-1, VP3-3, VP3-4, VP3-5, VP3-6, VP3-7, VP3-8, VP3-9, VP3-14, VP3-18, VP3-47 and any combination thereof, as shown in Table 2; and/or
(iv) an epitope of capsid protein VP4 selected from the group consisting of VP4-10, VP4-11, VP4-12 and any combination thereof, as shown in Table 2.

14. The anti-CVA16 IgY of claim 13, wherein the IgY specifically binds to
(i) an epitope of capsid protein VP1 selected from the group consisting of VP1-1, VP1-2, VP1-5, VP1-42, VP1-50 and any combination thereof,
(ii) an epitope of capsid protein VP2 selected from the group consisting of VP2-25; and/or
(iii) an epitope of capsid protein VP3 selected from the group consisting of VP3-1, VP3-4, VP3-5, VP3-6, VP3-7, VP3-8, VP3-9, VP3-14 and any combination thereof.

15. The anti-CVA16 IgY of claim 14, wherein the IgY does not bind to
(i) an epitope of capsid protein VP1 selected from the group consisting of VP1-6, VP1-14, VP-24, VP1-43 and any combination thereof;
(ii) an epitope of capsid protein VP2 selected from the group consisting of VP2-10, VP2-28, VP2-40 and any combination thereof,
(iii) an epitope of capsid protein VP3 selected from the group consisting of VP3-3, VP3-18, VP3-47 and any combination thereof; and/or
(iv) an epitope of capsid protein VP4 selected from the group consisting of VP4-10, VP4-11, VP4-12 and any combination thereof.

16. The anti-CVA16 IgY of claim 14-OF-15, wherein the anti-CVA16 IgY neutralizes CVA16 and enterovirus A71 (EV-A71).

17. The anti-CVA16 IgY of claim 12, wherein the IgY specifically binds to
(i) an epitope of capsid protein VP1 selected from the group consisting of VP1-1, VP1-6, VP1-14, VP-24, VP1-42, VP1-43 and any combination thereof;
(ii) an epitope of capsid protein VP2 selected from the group consisting of VP2-10, VP2-25, VP2-28, VP2-40 and any combination thereof;
(iii) an epitope of capsid protein VP3 selected from the group consisting of VP3-3, VP3-4, VP3-18, VP3-47 and any combination thereof; and/or
(iv) an epitope of capsid protein VP4 selected from the group consisting of VP4-10, VP4-11, VP4-12 and any combination thereof.

18. The anti-CVA16 IgY of claim 16, wherein the IgY does not bind to
(i) an epitope of capsid protein VP1 selected from the group consisting of VP1-2, VP1-5, VP1-50 and any combination thereof, and/or
(ii) an epitope of capsid protein VP3 selected from the group consisting of VP3-1, VP3-5, VP3-6, VP3-7, VP3-8, VP3-9, VP3-14 and any combination thereof.

19. The anti-CVA16 IgY of claim 17, wherein the anti-CVA16 IgY neutralizes CVA16 but does not neutralize enterovirus A71 (EV-A71).

20. A composition comprising the anti-CVA16 IgY of claim 12 and a carrier.

21. A method for treating or preventing CVA16 and/or EV-A71 infection, comprising administering to a subject in need thereof an effective amount of the anti-CVA16 IgY of claim 12 or a composition comprising the anti-CVA16 IgY and a carrier.

22. A method for diagnosing CVA16 and/or EV-A71 infection, comprising the steps of
(i) providing a sample derived from a subject in need thereof;
(ii) reacting the sample with the anti-CVA16 IgY of claim 12 or a composition comprising the anti-CVA16 IgY and a carrier; and
(iii) detecting an antigen-antibody reaction between the anti-CVA16 IgY and CVA16 and/or EV-A71 present in the sample.
